(19) United States
(12) Patent Application Publication
(10) Pub. No.: US 2013/0172407 A1
(43) Pub. Date: Jul. 4, 2013

(54) Inhibition of Cancer Cell Proliferation Using Oleoylethanolamide

(71) Applicant: China Medical University, Taichung City (TW)

(72) Inventors: Yueh-Hsiung Kuo, Taipei (TW); Feng-Yao Tang, Taichung City (TW); Hui-Yi Lin, Taichung City (TW)

(73) Assignee: China Medical University, Taichung City (TW)

(21) Appl. No.: 13/669,637

(22) Filed: Nov. 6, 2012

(30) Foreign Application Priority Data
Dec. 30, 2011 (TW) 100149822

(51) Int. Cl. A61K 31/164; A61P 35/00; A61K 31/202; A61K 31/336

(52) U.S. Cl. 514/475; 514/627; 514/560

(57) ABSTRACT

A pharmaceutical composition including oleoylethanolamide (OEA) is administered to inhibit tumor/cancer cell proliferation. The pharmaceutically composition may additionally include vitamin A, carotenoids, ω-3 polyunsaturated fatty acid, ω-6 polyunsaturated fatty acid and/or conjugated linolenic acid. The tumor/cancer may be colorectal cancer, lung adenocarcinoma, breast cancer, hepatoma, oral cancer and/or stomach adenocarcinoma.

### CROSS-REFERENCE TO RELATED APPLICATION

This application claims priority of Taiwanese patent application No. 100149822, filed on Dec. 30, 2011.

### BACKGROUND OF THE INVENTION

1. Field of the Invention

The present invention is directed to the inhibition of cancer cell proliferation using oleoylethanolamide (OEA).

2. Description of the Related Art

Cancer is one of the major causes that lead to human death. Although the mechanisms that result in cancer have yet to be completely understood, it is believed that the occurrence of cancer may be attributable to that when cells accumulate exogenous or endogenous factors that lead to genetic abnormalities, the signal transduction pathways in these cells may be aberrant, leading to uncontrolled cell cycle, and eventually, these cells would become cancer cells.

Apoptosis is considered a natural mechanism for regulating cell proliferation in animal cells. It plays important roles in the developmental processes of many cells, the maintenance of homeostasis and the elimination of damaged cells. Therefore, when cells become aberrant such that the mechanism of apoptosis loses control, these cells will proliferate abnormally and eventually become cancer cells.

In recent years, apoptosis and cell cycle control have become one of the major spotlights in oncology research. Researchers worldwide endeavor to develop anticancer drugs that can induce apoptosis of cancer cells and inhibit cell cycle progression of cancer cells. However, the medical effect achieved by the current clinical anticancer drugs remains unsatisfactory in cancer treatment. The major reasons include: individual diversity of patients themselves, severe side effects of anticancer drugs and drug resistance of cancer cells. Therefore, researchers in this field still work hard to develop useful drugs for treating cancer.

Many active ingredients obtained from animal or plant origins have been proven to exhibit anticancer effect, those of which include: vitamin A; carotenoids, such as lycopene, α-carotene, β-carotene, lutein, zeaxanthin, fucoxanthin, violaxanthin, astaxanthin, phytoene, phytofluene, neoxanthin and β-cryptoxanthin; ω-3 polyunsaturated fatty acid (PUFA), such as eicosapentaenoic acid (EPA) and docosahexaenoic acid (DHA); ω-6 polyunsaturated fatty acid, such as arachidonic acid (AA); and conjugated linolenic acid (CLA). (Dommels Y. E. et al. (2003), Carcinogenesis, 24:385-392; Calviello G. et al. (2004), Carcinogenesis, 25:2303-2310; H. Kohno et al. (2004), Cancer Sci., 95:481-486; Mauro M. et al. (2005), Anticancer Research, 25:3871-3876; Keren H. et al. (2007), Breast Cancer Res. Treat, 104:221-230; Palozza P. et al. (2008), Carcinogenesis, 29:2153-2161; F. Y. Tang et al. (2009), Journal of Nutritional Biochemistry, 20:426-434; Y. Satomi and H. Nishino (2009), Biochimica et Biophysica Acta, 1790:260-266; Liu C. et al. (2011), Cancer PreV. Res., 4:1255-1266; Xiao-dong S. et al. (2011), Biol. Pharm. Bull., 34:839-844; Virginie P. et al. (2011), Mar. Drugs, 9:819-831; Reynoso-Camacho R. et al. (2011), Nutr. Cancer, 63:39-45). Further report indicates that combination of lycopene and EPA can effectively inhibit proliferation of human colorectal cancer HT-29 cells in a synergistic way (F. Y. Tang et al. (2009), Journal of Nutritional Biochemistry, 20:426-434), in which inhibition of proliferation is reported as being, in part, associated with down-regulation of the PI-3K/Akt/mTOR signaling pathway. Active ingredients obtained from animal or plant origins are considered safer and do not generate undesired side effects in clinical applications, and thus, have become the focus in the field of modern pharmaceutics.

Acylethanolamides (AE), also called fatty acid ethanolamides (FAE), are a group of lipid signaling molecules widely found in plants and animals. When cells are subjected to various physiological or pathological stimuli, acylethanolamides would be released from cell membrane and regulate various cellular functions, including food intake, energy balance and cellular inflammation and proliferation, through signaling pathways. Currently found acylethanolamides include: anandamide, i.e., arachidonoylethanolamide (AEA); palmitoylethanolamide (PEA) and oleoylethanolamide (OEA).

It is known that anandamide is an endogenous ligand of cannabinoid receptor (CBR), which is a G protein-coupled receptor expressed on neurons and immune cells, and can stimulate food intake by activating cannabinoid receptor. In addition, it is reported that anandamide can inhibit the proliferation of colorectal carcinoma cells that express cyclooxygenase 2 (COX-2) and induce non-apoptotic cell death through metabolism of anandamide by COX-2, and thus, may be a useful chemopreventive/therapeutic agent for colorectal cancer cells that highly express COX-2 or tumor cells that have become resistant to apoptosis (H. A. Patsos et al. (2005), Gut, 54:1741-1750).

Oleoylethanolamide (OEA) is a monounsaturated analogue of anandamide, and has the structure of the following chemical formula (i):

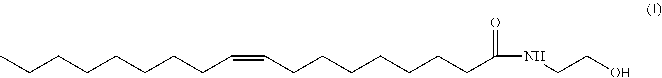 [SMILES: CCCCCCCC/C=C\CCCCCCCC(=O)NCCO]

Unlike anandamide, OEA has only one double bond and does not bind to cannabinoid receptor. Rather, it induces satiety and inhibits weight gain while stimulating fat utilization, such as lipolysis and fatty acid oxidation, by activating peroxisome proliferator-activated receptor a (PPAR-α). Therefore, OEA can be provided for treating eating disorder and obesity (Jin Fu et al. (2003), Nature, 425:90-93; Manuel Guzman et al. (2004), The Journal of Biological Chemistry, 27:27849-27854).

Surprisingly, it is found by the inventors of this invention that OEA, in addition to the anorexiant and lipolytic effects, may also be able to induce cell cycle arrest and apoptosis of cancer cells. Particularly, combined use of OEA and lycopene may synergistically inhibit proliferation of cancer cells. Thus, OEA and its combination with other active ingredients, such as lycopene, are expected to be useful in cancer treatment.

### SUMMARY OF THE INVENTION

The present invention provides a method for inhibiting tumor/cancer cell proliferation, comprising administering to a patient in need thereof a pharmaceutical composition comprising oleoylethanolamide. Oleoylethanolamide may inhibit tumor/cancer cell proliferation by inducing apoptosis and cell cycle arrest of the tumor/cancer cells. In addition, oleoylethanolamide may be combined with other active ingredients, such as lycopene, to provide synergistic effect in inhibiting tumor/cancer cell proliferation.

### BRIEF DESCRIPTION OF THE DRAWING

Other features and advantages of the present invention will become apparent in the following detailed description of the preferred embodiments of this invention, with reference to the accompanying drawings, in which:

FIG. 1 shows the cell viability percentage detected after HT-29 cells were treated with different concentrations of OEA and lycopene, alone or in combination, wherein “*” represents p<0.05;

FIG. 2 shows the cell viability percentage detected after HCT 116 cells were treated with different concentrations of OEA and lycopene, alone or in combination, wherein “*” represents p<0.05;

FIG. 3 shows the cell viability percentage detected after SW-480 cells were treated with different concentrations of OEA and lycopene, alone or in combination, wherein “*” represents p<0.05;

FIG. 4 shows the cell viability percentage detected after A549 cells were treated with different concentrations of OEA and lycopene, alone or in combination, wherein “*” represents p<0.05;

FIG. 5 shows the cell viability percentage detected after MDA-MB-231 cells were treated with different concentrations of OEA and lycopene, alone or in combination, wherein “*” represents p<0.05;

FIG. 6 shows the cell viability percentage detected after Huh-7 cells were treated with different concentrations of OEA and lycopene, alone or in combination, wherein “*” represents p<0.05;

FIG. 7 shows the cell viability percentage detected after CAL 27 cells were treated with different concentrations of OEA and lycopene, alone or in combination, wherein “*” represents p<0.05;

FIG. 8 shows the cell viability percentage detected after AGS cells were treated with different concentrations of OEA and lycopene, alone or in combination, wherein “*” represents p<0.05;

FIG. 9 shows the percentage of cells in the Sub-G1 phase after HT-29 cells were treated with different concentrations of OEA and lycopene, alone or in combination, wherein “*” represents p<0.05;

FIG. 10 is a picture of Western Blotting Analysis showing the expression profile of cyclin D1 and p-NF-κB p65 detected after HT-29 cells were treated with different concentrations of OEA and lycopene, alone or in combination; and

FIG. 11 shows the cell death percentage detected after HT-29 cells were treated with different concentrations of OEA and lycopene, alone or in combination, wherein “*” represents p<0.05.

### DETAILED DESCRIPTION OF THE PREFERRED EMBODIMENTS

It is to be understood that, if any prior art publication is referred to herein, such reference does not constitute an admission that the publication forms a part of the common general knowledge in the art, in Taiwan or any other country.

For the purpose of this specification, it will be clearly understood that the word “comprising” means “including but not limited to”, and that the word “comprise” has a corresponding meaning.

Unless otherwise defined, all technical and scientific terms used herein have the meaning commonly understood by a person skilled in the art to which this invention belongs. One skilled in the art will recognize many methods and materials similar or equivalent to those described herein, which could be used in the practice of this invention. Indeed, this invention is in no way limited to the methods and materials described.

In developing drugs for treating tumor/cancer cells, the inventors found that oleoylethanolamide (OEA) may inhibit tumor/cancer cell proliferation by inducing apoptosis and cell cycle arrest of the tumor/cancer cells. Therefore, the present invention provides a method for inhibiting tumor/cancer cell proliferation by administering to a patient in need thereof a pharmaceutical composition comprising OEA.

Particularly, by in vitro anticancer cell test, the inventors demonstrated that both OEA and its combination with lycopene exhibit cytotoxicity to various cancer cell lines, including cell lines of colorectal cancer, lung adenocarcinoma, breast cancer, hepatoma, oral cancer and stomach adenocarcinoma, and that the combined use of OEA and lycopene may synergistically inhibit proliferation of these cancer cell lines. In addition, both OEA and its combination with lycopene may induce apoptosis of cancer cells, promoting death of cancer cells, and thus, achieving the effect of inhibiting tumor/cancer cell proliferation. Therefore, OEA and its combination with other active ingredients, such as lycopene, may have potency in developing into an anticancer drug.

According to the present invention, the tumor/cancer cells are selected from the group consisting of colorectal cancer cells, lung adenocarcinoma cells, breast cancer cells, hepatoma cells, oral cancer cells, stomach adenocarcinoma cells and combinations thereof.

According to the present invention, the pharmaceutical composition may further comprise an active ingredient. Preferably, the active ingredient is selected from the group consisting of: vitamin A; carotenoids; ω-3 polyunsaturated fatty acid, such as eicosapentaenoic acid (EPA) and docosahexaenoic acid (DHA); ω-6 polyunsaturated fatty acid, such as arachidonic acid (AA); conjugated linolenic acid; and combinations thereof.

According to the present invention, the carotenoid is selected from the group consisting of lycopene, α-carotene, β-carotene, lutein, zeaxanthin, fucoxanthin, violaxanthin, astaxanthin, phytoene, phytofluene, neoxanthin, β-cryptoxanthin and combinations thereof. In a preferred embodiment of this invention, the active ingredient is lycopene.

According to the present invention, OEA and the active ingredient may be mixed or separately prepared into a pharmaceutically acceptable composition followed by packaging into a dosage form suitable for administration.

According to the present invention, the pharmaceutical composition according to this invention may be formulated into a suitable dosage form for parenteral, topical or oral administration using technology well known to those skilled in the art, which includes, but is not limited to, injection such as sterile aqueous solution or dispersion, sterile powder, tablet, troche, pill, capsule, and the like.

The pharmaceutical composition according to this invention may be administered through a parenteral route selected from the group consisting of intraperitoneal injection, subcutaneous injection, intramuscular injection and intravenous injection.

Preferably, the pharmaceutical composition according to this invention is formulated into a dosage form suitable for subcutaneous injection.

Preferably, the pharmaceutical composition according to this invention is formulated into a dosage form suitable for oral administration.

The pharmaceutical composition according to this invention may additionally comprise a pharmaceutically acceptable carrier widely employed in the art of drug-manufacturing. For instance, the pharmaceutically acceptable carrier may include one or more of the agents selected from the group consisting of solvents, emulsifiers, suspending agents, decomposers, binding agents, excipients, stabilizing agents, chelating agents, diluents, gelling agents, preservatives, lubricants, absorption delaying agents, liposomes, and the like.

Examples of the solvent include water, normal saline, phosphate buffered saline (PBS), a sugar-containing solution, an aqueous solution containing alcohol, and combinations thereof. In a preferred embodiment of this invention, the solvent is an aqueous solution containing alcohol.

The present invention further provides a method for treating a subject having or suspected of having cancer, comprising administering to the subject OEA.

According to the method of the present invention, OEA may be further combined with one of the active ingredients as described above and administered to the patient in need thereof. OEA and the active ingredient may be separately or simultaneously administered to the patient.

According to the present invention, the dosage and administration intervals of the pharmaceutical composition of this invention may depend on the following factors: the severity of the disease to the treated, the administration route and the weight, age, body condition and response of the subject to be treated. In general, the daily dosage of OEA in the present invention is 0.3 mg/Kg to 1.5 mg/Kg, administered in a form of single dosage or multiple dosage and may be orally or parenterally administered.

This invention will be further described by way of the following examples. However, it should be understood that the following examples are solely intended for the purpose of illustration and should not be construed as limiting the invention in practice.

### EXAMPLES

### Experimental Materials

### Source and Culture of Cell Lines:

The species, sources and deposit numbers of cell lines used in the following examples are shown in Table 1 below, and the cell lines were purchased from American Type Culture Collection (ATCC) or Japan Collection of the Research Biosources (JCRB).

TABLE 1

| | | Deposit |
|---|---|---|
| Names of Cell Lines | Source | Number |
| Human Colorectal Cancer Cell HT-29 | ATCC | HTB-38 |
| Human Colorectal Cancer Cell HCT 116 | ATCC | CCL-247 |
| Human Colorectal Cancer Cell SW-480 | ATCC | CCL-228 |
| Human Lung Adenocarcinoma Cell A549 | ATCC | CCL-185 |
| Human Breast Cancer Cell MDA-MB-231 | ATCC | HTB-26 |
| Human Hepatoma Cell Huh-7 | JCRB | 403 |
| Human Oral Cancer Cell CAL 27 | ATCC | CRL-2095 |
| Human Stomach Adenocarcinoma Cell AGS | ATCC | CRL-1739 |

HT-29 cells were cultured in a 75-cm² flask containing McCoy's medium (Invitrogen Inc., Carlsbad, Calif., USA) supplemented with 10% Fetal Bovine Serum (FBS), 2 mM glutamine and 1.5 g/L sodium bicarbonate. The remaining cells were cultured in a 75-cm² flask containing RPMI 1640 medium (Hyclone, Grand Island, N.Y., USA) supplemented with 10% Fetal Bovine Serum (FBS), 2 mM glutamine and 1.5 g/L sodium bicarbonate. These cells were cultured in an incubator with culture conditions set at 37° C. and 5% CO₂.

### General Experimental Procedure:

### Statistical Analysis:

In the following examples, experiments in each group were repeated 3 times. The experimental data are expressed as “mean±standard error of the mean (SEM).” All the data were analyzed using one-way analysis of variance (one-way ANOVA) followed by post hoc test, so as to evaluate the difference between the groups. If the obtained result of the statistical analysis is p<0.05, this represents statistical significance.

### Example 1

### Preparation of Oleoylethanolamide (OEA)

1 mL DMF, 99.7 mg oleic acid and 80 μL triethylamine were mixed followed by stirring at room temperature for 2 minutes to obtain a mixture. Then, N-ethanolamine (122 μL, 1.0 mmol) and Benzotriazole-1-yl-oxy-tris-(dimethylamino)-phosphonium hexafluorophosphate (BOP) (243.8 mg) were dissolved in CH₂Cl₂ (1 mL) and then added to the mixture and stirred for 30 minutes under 0° C. to obtain a reaction mixture. The reaction mixture was left to stand at room temperature and then stirred overnight. The reaction mixture was then concentrated under a reduced pressure at 50° C. so as to remove solvent. Subsequently, the resultant residue was purified using silicone column chromatography (n-hexane/EtOAc=1:1) to obtain a compound in a colorless liquid.

The purified compound was subjected to high-resolution electron impact mass spectrometry (HREIMS) using a Finnigan TSQ-46C mass spectrometer. The detected experimental data was as follows: calculated value for C₂₀H₃₉O₂N [M+1]⁺; 326.2971. found value: 326.2974.

According to the spectrometry data thus detected, the compound was confirmed to be oleoylethanolamide (OEA) having the following chemical structure:

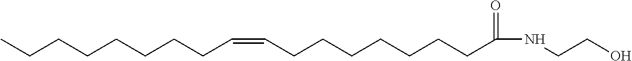 [SMILES: CCCCCCCC/C=C\CCCCCCCC(=O)NCCO]

### Example 2

### In Vitro Anti-Cancer Test of OEA and its Combination with Lycopene

### A. Preparation of OEA Solution, Lycopene Solution and Combined Solution:

Lycopene (Extrasynthese, Genay, France) and OEA obtained from the above Example 1 were dissolved in tetrahydrofuran (THF) containing 0.025% butylated hydroxytoluene (BHT) as an antioxidant and ethanol, respectively, to formulate a 10 mM lycopene stock solution and a 400 mM OEA stock solution.

Appropriate amounts of lycopene stock solution and OEA stock solution were mixed followed by adding FBS and mixing for 30 minutes to obtain a combined solution. Then, a serum-free cell culture medium, tetrahydrofuran and ethanol were used to adjust concentrations of the OEA stock solution, the lycopene stock solution, and combined solution, thereby formulating different concentrations of OEA solutions (i.e., 50, 100 and 200 μM) and lycopene solutions (i.e., 2 and 5 μM) as well as combined solution 1 (comprising 200 μM of OEA and 2 μM of lycopene) and combined solution 2 (comprising 200 μM of OEA and 5 μM of lycopene), respectively. The formulations of the diluted solutions are shown in Table 2 below.

TABLE 2

| | | Lycopene | Combined | Combined |
|---|---|---|---|---|
| Components | OEA solution | solution | solution 1 | solution 2 |
| FBS (%) (v/v) | 10 | 10 | 10 | 10 |
| ethanol (%) (v/v) | 0.05 | 0.05 | 0.05 | 0.05 |
| Tetrahydrofuran | 0.05 | 0.05 | 0.05 | 0.05 |
| (%) (v/v) | | | | |
| OEA (μM) | 50, 100 or | — | 200 | 200 |
| | 200 | | | |
| Lycopene (μM) | — | 2 or 5 | 2 | 5 |

The remaining is the serum-free cell culture medium

In the examples below, cell experimental groups treated with the OEA solutions are called “OEA groups,” the cell experimental groups treated with the lycopene solutions are called “lycopene groups,” and the cell experimental groups treated with the combined solutions are called “synergy groups.”

B. Treatment of Various Cancer Cell Lines with OEA Solutions, Lycopene Solutions and Combined Solutions:

Each of HT-29 cells, HCT 116 cells, SW-480 cells, A549 cells, MDA-MB-231 cells, Huh-7 cells, CAL 27 cells and AGS cells was divided into 8 groups including 1 control group and 7 experimental groups (i.e., OEA groups 1 to 3, lycopene groups 1 and 2 and synergy groups 1 and 2). The cells of each group were add to a 24-well plate (5×10⁴ cells/well) containing 1 mL cell culture medium (the culture medium used for each cell line was that described in “Experimental material”) and then incubated in an incubator (37° C., 5% CO₂) for 24 hours.

Subsequently, the medium in each group was removed, and then the OEA solutions, the lycopene solutions and the combined solutions obtained from Section A of this example were added to the respective experimental groups (see Table 3 below). The control group was added equal volume of the cell culture medium (supplemented with 10% (v/v) FBS, 0.05% (v/v) tetrahydrofuran and 0.05% (v/v) ethanol).

TABLE 3

| Experimental | | Amount |
|---|---|---|
| group | Solution | (mL) |
| OEA group 1 | 50 μM OEA solution | 0.5 |
| OEA group 2 | 100 μM OEA solution | 0.5 |
| OEA group 3 | 200 μM OEA solution | 0.5 |
| Lycopene | 2 μM lycopene solution | 0.5 |
| group 1 | | |
| Lycopene | 5 μM lycopene solution | 0.5 |
| group 2 | | |
| Synergy group 1 | Combined solution 1 | 0.5 |
| | (comprising 200 μM OEA and 2 μM | |
| | lycopene) | |
| Synergy group 2 | Combined solution 2 | 0.5 |
| | (comprising 200 μM OEA and 5 μM | |
| | lycopene) | |

Subsequently, the cells in each group were incubated in an incubator (37° C., 5% CO₂) for 24 hours. The obtained cell culture was used for the following cell viability analysis.

### C. Cell Viability Analysis:

{3-[4,5-Dimethylthiazol-2-yl]-2,5-diphenyltetrazolium bromide} (MTT, 0.5 mg/mL, 200 μL) was added to the cell culture of each group, followed by incubation in an incubator (37° C., 5% CO₂) for 2 hours. Then, the liquid in each well was removed and 200 μL acidic isopropanol was added, followed by mixing homogeneously. The absorbance at 570 nm (OD₅₇₀) for each well was detected using a microplate reader (Tecan Inc., Mannedorf, Switzerland).

Cell viability percentage (%) was calculated by substituting the detected absorbance (OD₅₇₀) to the following formula (1):

A=(B/C)×100 Formula (1):
wherein:
   A=cell viability percentage (%)
   B=the detected OD₅₇₀ absorbance of each group
   C=the detected OD₅₇₀ absorbance of control group

Then, the obtained experimental data were analyzed according to the method described in “Statistical Analysis” in the section of “General experimental procedure.”

### Results:

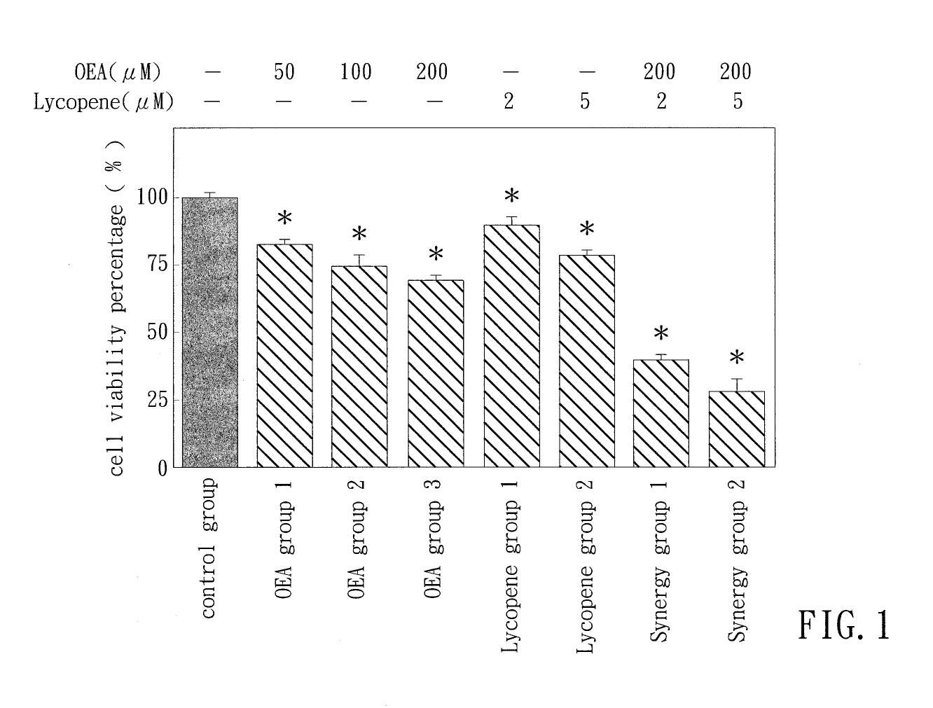

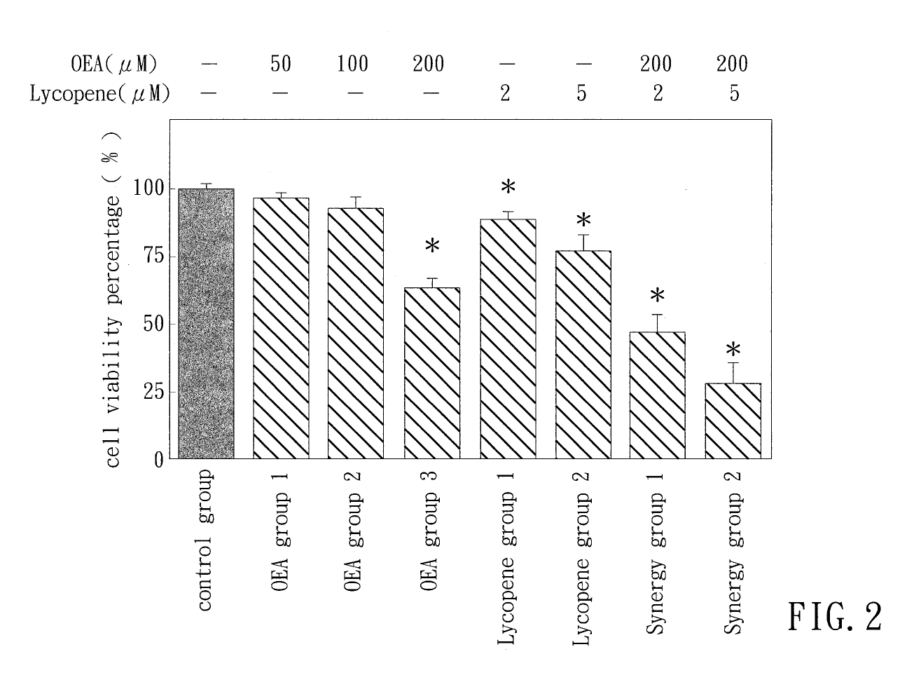

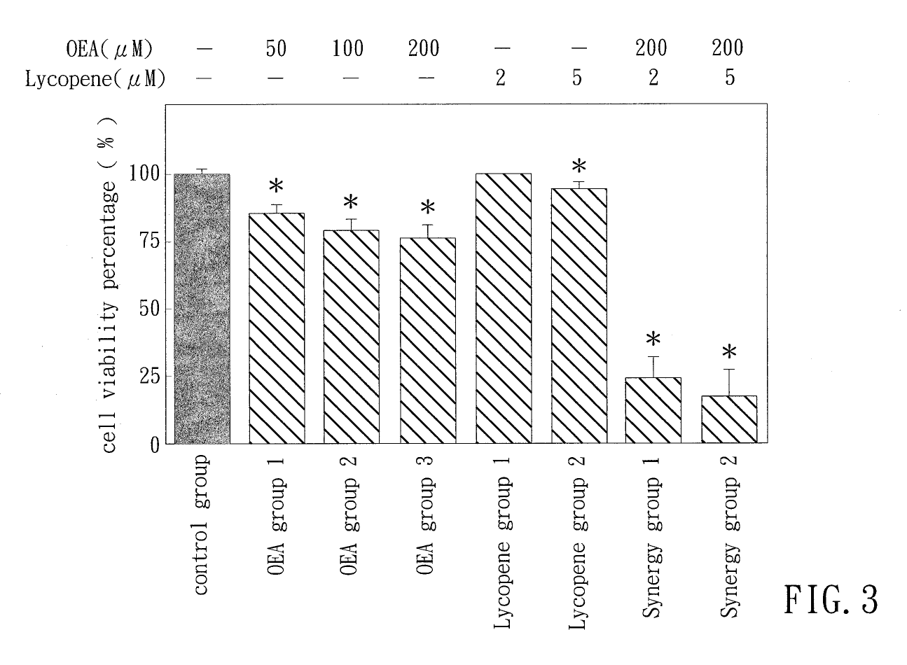

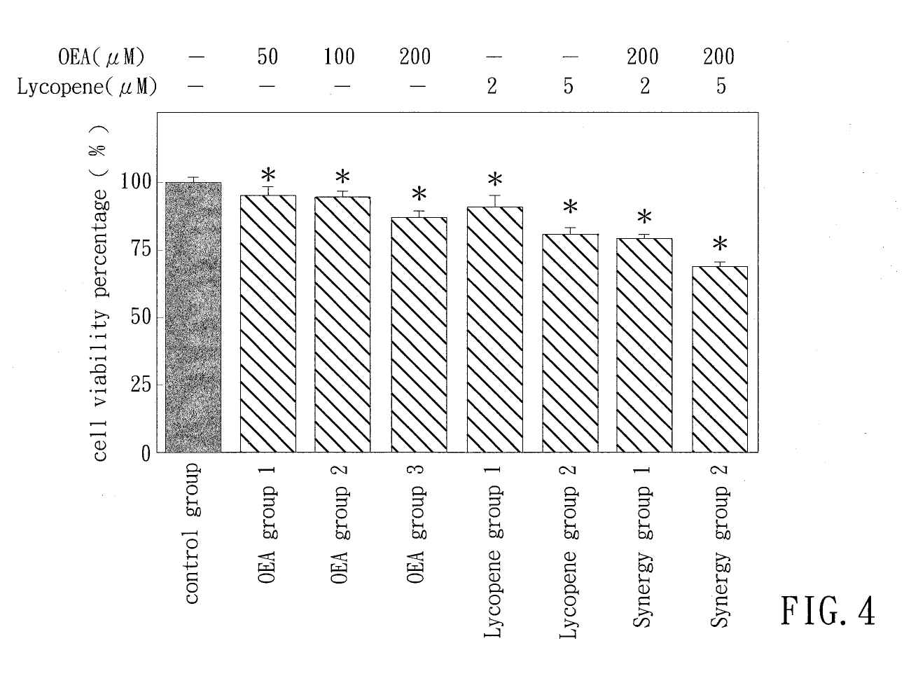

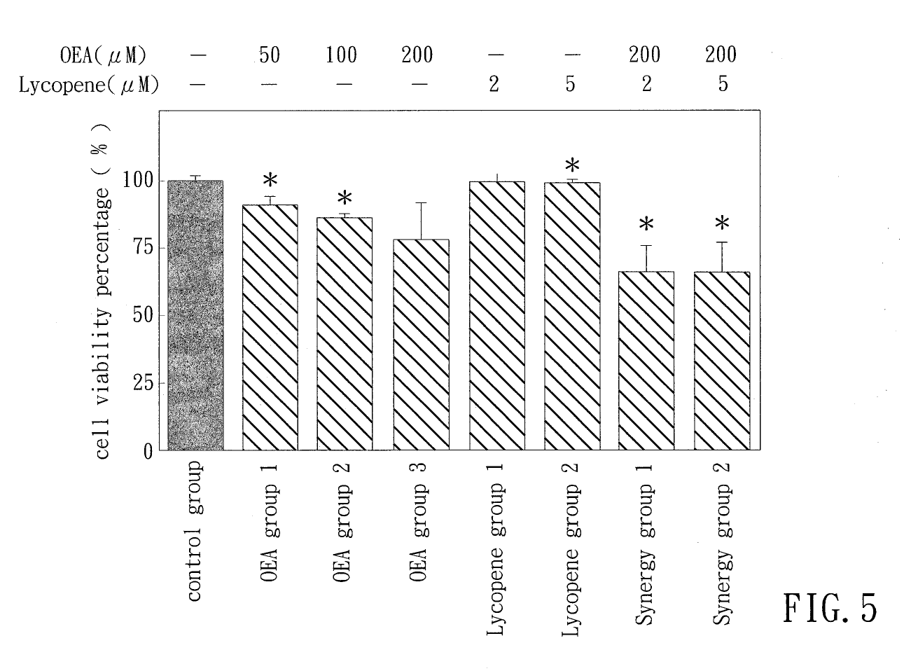

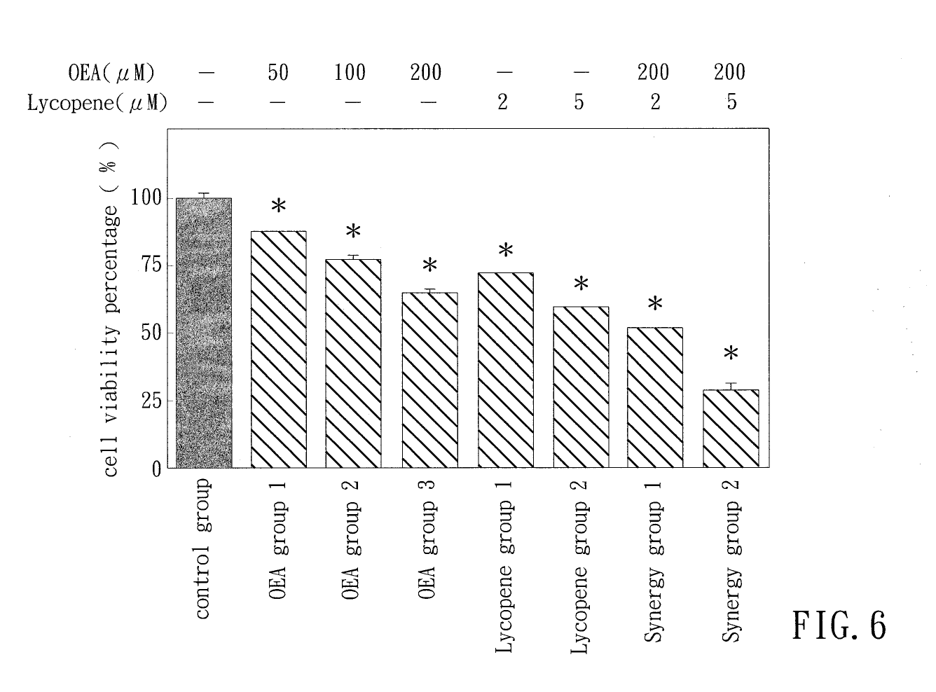

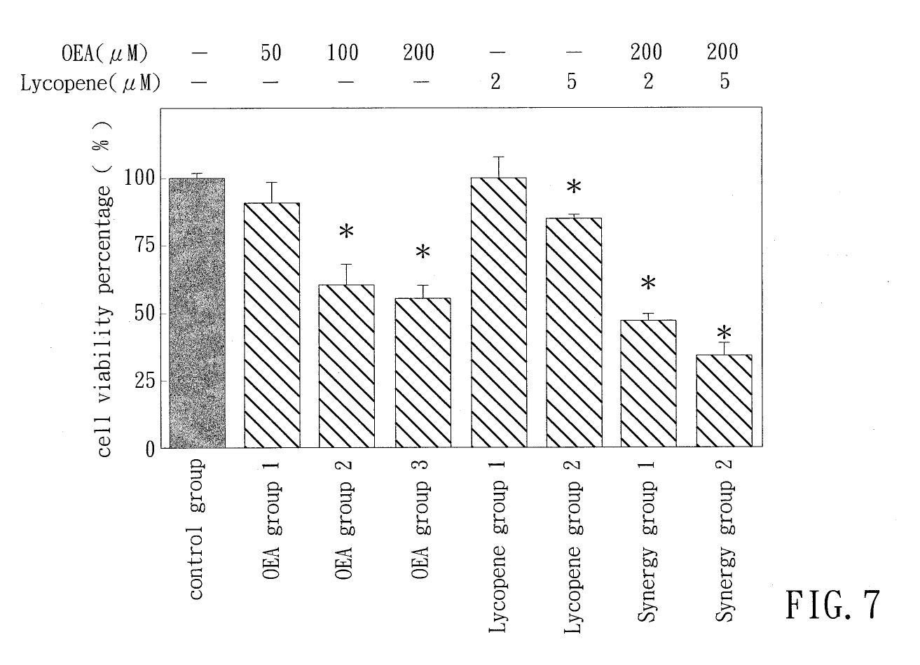

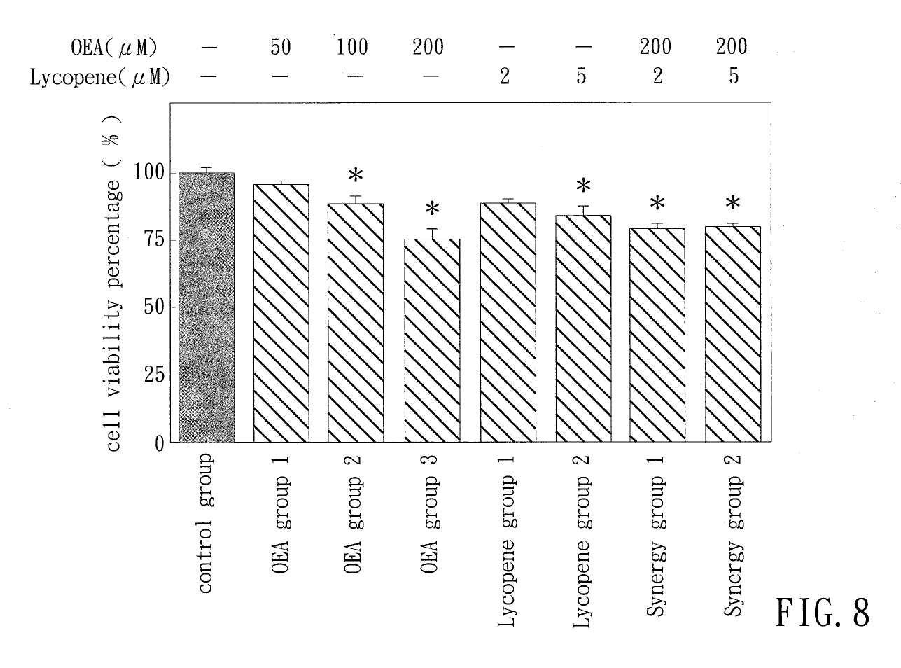

FIGS. 1 to 8 show the detected cell viability percentage after HT-29 cells, HCT 116 cells, SW-480 cells, A549 cells, MDA-MB-231 cells, Huh-7 cells, CAL 27 cells and AGS cells were treated with different concentrations of OEA and lycopene, alone or in combination, respectively. From FIGS. 1 to 8, it is shown that, compared to the control group, the cell viability percentage of each cancer cell line in OEA groups 1 to 3 and lycopene groups 1 to 2 is reduced, and such reduction becomes apparent as the concentration of OEA or lycopene increases. In addition, compared to OEA group 3 and lycopene groups 1 and 2, the cell viability percentage in synergy groups 1 and 2 is reduced more greatly.

The experimental results show that OEA and lycopene exhibit cytotoxicity to cell lines of all the cancers (including colorectal cancer, lung adenocarcinoma, breast cancer, hepatoma, oral cancer and stomach adenocarcinoma) and have a positive dose-effect relationship. Further, combined use of OEA and lycopene exhibits synergistic effect in inhibiting survival of these cancer cell lines (particularly colorectal cancer cell line SW-480). Therefore, the Applicants of this invention suggest that OEA and its combination with lycopene exhibit activity of inhibiting cancer cell proliferation and thus may be used for treating cancer, particularly colorectal cancer. According to this result, the Applicants of this invention chose colorectal cancer cell for subsequent experiments.

### Example 3

### Effect of OEA and its Combination with Lycopene on the Cell Cycle and Proliferation of Human Colorectal Cancer Cell Line HT-29

In order to understand the effect of OEA and its combination with lycopene on the cell cycle and proliferation of human colorectal cancer cell line HT-29, HT-29 cell culture treated with different solutions obtained from Item B of Example 2 was used for the following experiments.

### A. Flow Cytometry:

Each group of the HT-29 cell culture obtained from Item B of Example 2 was washed with PBS twice followed by fixing the cells with 70% (w/v) cold ethanol for 1 hour. Then, the obtained fixed cells were washed with cold PBS followed by centrifugation at 300 g for 5 minutes. The supernatant was removed and 1 mL cold DNA dying solution which was prepared in PBS and containing 200 μg/mL RNase A solution and 200 μg/mL propidium iodide (PI) was added to re-suspend the cells in each group. The cells were kept in dark for 30 minutes at room temperature to obtain dyed cells.

Subsequently, the obtained dyed cells were subjected to cell cycle analysis using BD FACSCanto™ flow cytometer (BD Biosciences, Cat. No. 338960), and the DNA content of 1×10⁴ cells was analyzed in each analysis. The cells would emit fluorescence when excited by laser beam of argon ion at 488 nm. Cell cycle profile was analyzed from DNA content histograms using BD FACSDiVa™ software (Becton Dickinson, Cat. No. 643629). When the cells are undergoing apoptosis, DNA contained in these cells would be digested by endonucleases, thereby occurring a Sub-G1 peak. The percentage of the cells in Sub-G1 phase was analyzed using ModFit LT software.

The obtained experimental data was analyzed according to the method described in “Statistical Analysis” in the section of “General experimental procedure”.

B. Expression Profile of Proteins Associated with Cell Cycle Progression:

In order to detect the protein expression of HT-29 cells, the HT-29 cell culture treated with different solutions for use in the instant experiment was obtained substantially according to “B. Treatment of various cancer cell lines with OEA solutions, lycopene solutions and combined solutions” in the above Example 2. The difference was that the cells of each group were cultured in a culture dish having a diameter of 10 cm. Experimental groups were added with different solutions according to those shown in Table 3 and the amount added was 10 mL.

Then, the HT-29 cell cultures of the control group, OEA group 3, lycopene groups 1 and 2 and synergy groups 1 and 2 were washed with PBS twice followed by adding 75 μL lysis buffer (containing PBS, 1% Ipegal CA-630 (Sigma, St. Louis, Mo.), 0.5% sodium deoxycholate, 0.1% SDS, 100 μM phenylmethylsulfonyl fluoride, 20 μg/mL aprotinin, 1 mM PMSF and 3 μM sodium orthovanadate) and uniformly mixing. The resultant cell mixtures were subjected to nuclear protein extraction using NE-PER® Nuclear and Cytoplasmic Extraction Reagents (Thermo Scientific, Cat. No. 78833) according to the manufacturer's operation guide, thereby obtaining nuclear protein samples.

The nuclear protein samples were subjected to sodium dodecyl sulfate-polyacrylamide gel electrophoresis (SDS-PAGE) analysis according to the technique known and routinely used by one skilled in the related art, and to Western Blotting analysis for cyclin D1 and p-NF-κB p65 while Lamin A/C was used as an internal control. The instruments and reagents used were as follows:

(1) SDS-PAGE analysis was performed using a vertical electrophoresis cell (BioRAD Mini-PROTEAN® 3 Cell).
(2) Protein transfer was performed using an electrophoresis transfer cell (BioRAD Mini Trans-Blot® Cell) and a nitrocellulose membrane.
(3) In Western Blotting analysis, primary and secondary antibodies used for detecting each protein are shown in Table 4 below.

TABLE 4

| Protein | Primary Antibody | Secondary Antibody |
|---|---|---|
| p-NF-κB | Rabbit anti phospho-NF-κB p65 | Goat anti rabbit |
| p65 | monoclonal antibody (Cell | IgG-peroxidase antibody |
| | signaling, Cat. No. #3033) | (Sigma, Cat. No. A0545) |
| Cyclin D1 | Mouse anti cyclin D1 monoclonal | Goat anti-mouse IgG- |
| | antibody (BD Pharmingen ™, Cat. | horseradish peroxidase |
| | No. 556470) | (HRP) antibody (Santa |
| | | Cruz, Cat. No. sc-2005) |
| Lamin | Rabbit anti Lamin A/C polyclonal | Goat anti rabbit |
| A/C | antibody (Cell signaling, Cat. No. | IgG-peroxidase antibody |
| | #2032) | (Sigma, Cat. No. A0545) |

(4) Chemiluminescence staining was performed using Western Lightning® Plus-ECL (Perkin Elmer, Cat. No. NEL105), and Fujifilm LAS-4000 (Fujifilm Life Science, USA) was used to detect signal.

### Results:

### A. Flow Cytometry

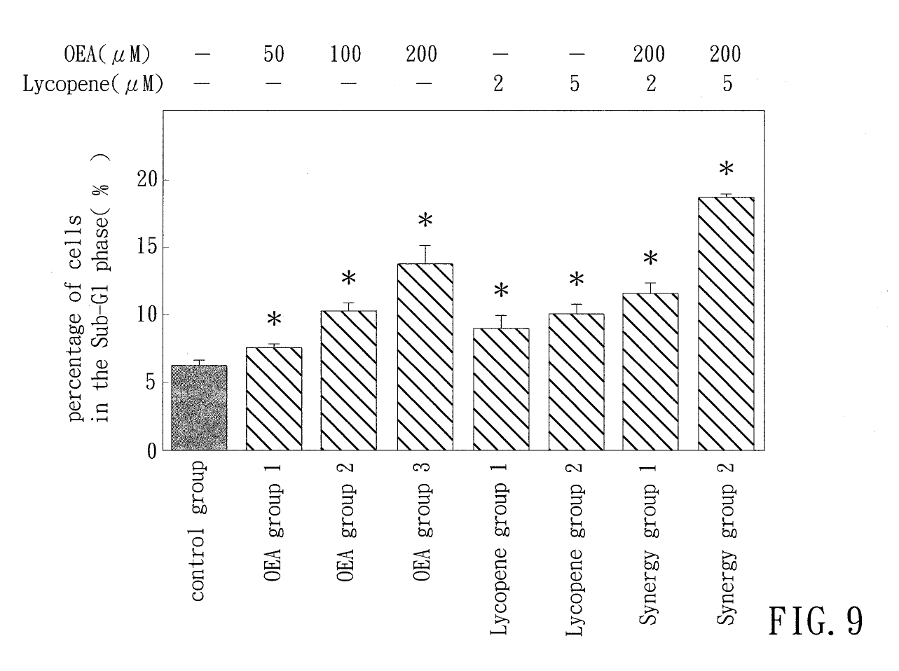

FIG. 9 shows the percentage of HT-29 cells in Sub-G1 phase after the cells were respectively treated with different concentrations of OEA and lycopene, alone or in combination. As shown in FIG. 9, both OEA and lycopene promote HT-29 cells to be in Sub-G1 phase, and this becomes more apparent as the concentration of OEA or lycopene increases. Particularly, compared to OEA groups 1-3 and lycopene groups 1 and 2, in synergy groups 1 and 2, especially synergy group 2, more HT-29 cells were in Sub-G1 phase.

This experimental results show that OEA and its combination with lycopene can induce cell cycle arrest for HT-29 cells and that this induction exhibits a positive dose-effect relationship.

B. Expression Profile of Proteins Associated with Cell Cycle Progression

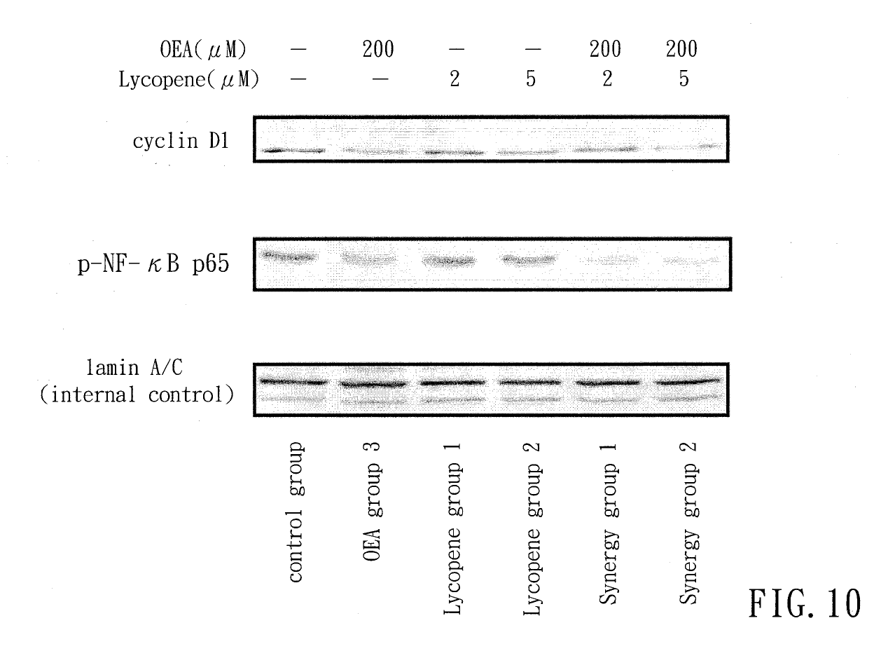

FIG. 10 is a picture of Western Blotting analysis showing the expression profile of cyclin D1 and p-NF-κB p65 detected after HT-29 cells were respectively treated with different concentrations of OEA and lycopene, alone or in combination. As shown in FIG. 10, compared to the control group, the amount of cyclin D1 and p-NF-κB p65 expression of the HT-29 cells in OEA group 3 and lycopene groups 1 and 2 are reduced. Further, compared to OEA group 3 and lycopene groups 1 and 2, the amount of cyclin D1 and p-NF-κB p65 expression of the HT-29 cells in synergy groups 1 and 2 are greatly reduced.

This experimental results show that OEA and its combination with lycopene can reduce the expression of cyclin D1 and p-NF-κB p65 in colorectal cancer cell line HT-29. The results reveal that OEA and its combination with lycopene can promote cell cycle arrest of the cancer cells by regulating the expression of proteins that are associated with cell cycle progression in the cancer cells, thereby achieving inhibition of cancer cell proliferation.

### Example 4

### Effect of OEA and its Combination with Lycopene on Cell Apoptosis of Human Colorectal Cancer Cell Line HT-29

In order to further investigate the usefulness of OEA and its combination with lycopene in inducing cell apoptosis of human colorectal cancer cell line HT-29, HT-29 cell cultures of control group, OEA group 3, lycopene groups 1 and 2 and synergy groups 1 and 2 obtained according to the above Item B in Example 2 were used for subsequent experiments.

First, the cell culture of each group was collected and washed with cold PBS twice followed by centrifugation at 300 g for 5 minutes. The supernatant was removed and 1× Annexin V Binding Buffer (BD Pharmingen™) was added to sufficiently re-suspend the cell pellet thereby obtaining a cell suspension having a cell concentration of 1×10⁵ cells/mL. Then, 5 μL FITC Annexin V (BD Pharmingen™) and 5 μL Propidium Iodide (PI) staining solution (BD Pharmingen™) were added to the obtained cell suspension, followed by mixing homogeneously and standing in dark at room temperature for 15 minutes to obtain stained cells.

Then, the stained cells were analyzed using BD FACSAria™ flow cytometry, wherein cells stained with FITC Annexin V and PI, i.e., FITC Annexin V positive and PI positive, represent cells that were induced with apoptosis and died, while cells that could not be stained with FITC Annexin V and PI, i.e., FITC Annexin V negative and PI negative, represent viable cells. The number of cells stained with FITC Annexin V and PI was calculated using BD FACSDiVa™ software.

The cell death percentage (%) of each group was calculated by substituting the detected cell number to the following formula (2):

D=(E/F)×100 Formula (2):
wherein:
   D=cell death percentage (%)
   E=the number of cells stained with FITC Annexin V and PI
   F=total cells

The obtained experimental data were analyzed according to the method described in “Statistical Analysis” in the section of “General experimental procedure.”

### Results:

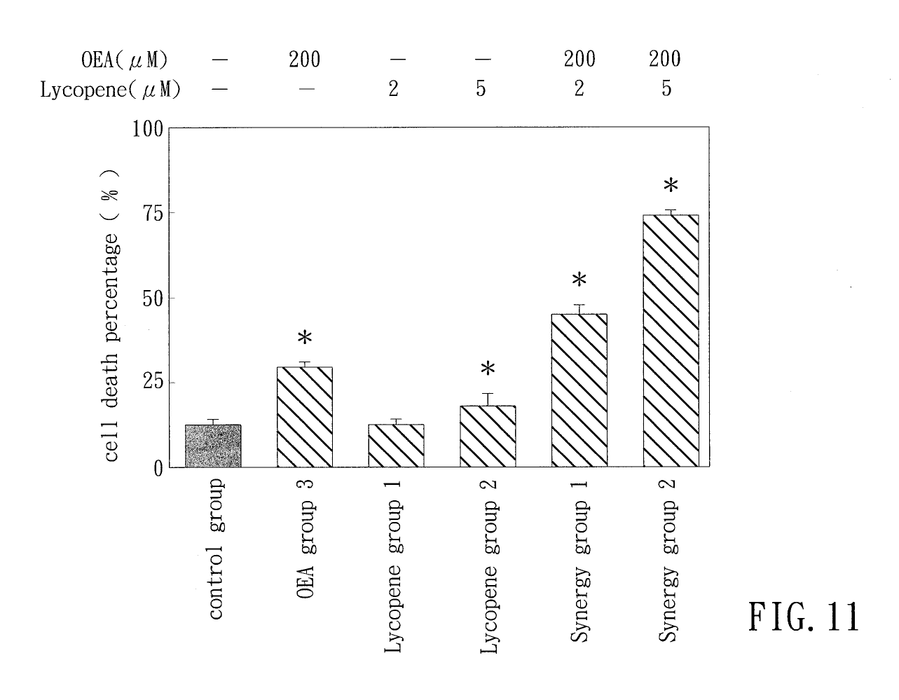

FIG. 11 shows the cell death percentage detected after HT-29 cells were treated with different concentrations of OEA and lycopene, alone or in combination. As shown in FIG. 11, compared to the control group, cell death percentage in OEA group 3 and lycopene group 2 are elevated. Further, compared to OEA group 3 and lycopene group 2, cell death percentage in synergy groups 1 and 2 are greatly elevated.

This experimental results show that OEA and its combination with lycopene can induce HT-29 cells to undergo the mechanism of cell apoptosis, thereby promoting death of the cancer cells and inhibiting proliferation of the cancer cells. Particularly, OEA and its combined use with lycopene exhibit synergistic effect in inducing HT-29 cell apoptosis which demonstrates superior anti-cancer activity. Therefore, OEA and its combination with lycopene might exhibit high potency in developing into an anti-cancer drug.

All the patents and references cited in this specification are incorporated herein in their entirety as reference. When there is conflict, the detailed descriptions in this case, including the definitions, would prevail.

While the present invention has been described with reference to the above particular embodiments, many modifications and changes can be made without apparently deviating from the scope and spirit of the present invention.

What is claimed is:

1. A method for inhibiting tumor/cancer cell proliferation, comprising administering to a patient in need thereof a pharmaceutical composition comprising oleoylethanolamide (OEA).

2. The method of claim 1, wherein the tumor/cancer is selected from the group consisting of colorectal cancer, lung adenocarcinoma, breast cancer, hepatoma, oral cancer, stomach adenocarcinoma and combinations thereof.

3. The method of claim 2, wherein the tumor/cancer is colorectal cancer.

4. The method of claim 1, wherein the pharmaceutical composition further comprises an active ingredient selected from the group consisting of vitamin A, carotenoids, ω-3 polyunsaturated fatty acid, ω-6 polyunsaturated fatty acid, conjugated linolenic acid and combinations thereof.

5. The method of claim 4, wherein the carotenoid is selected from the group consisting of lycopene, α-carotene, β-carotene, lutein, zeaxanthin, fucoxanthin, violaxanthin, astaxanthin, phytoene, phytofluene, neoxanthin, β-cryptoxanthin and combinations thereof.

6. The method of claim 5, wherein the carotenoids is lycopene.

7. The method of claim 1, wherein the inhibiting tumor/cancer cell proliferation is through cell cycle arrest.

8. The method of claim 7, wherein expression of cyclin D1 and p-NF-κB p65 are reduced.

9. The method of claim 1, wherein the inhibiting tumor/cancer cell proliferation is through apoptosis.

10. The method of claim 1, wherein the pharmaceutical composition further comprises a pharmaceutically acceptable carrier.

11. The method of claim 10, wherein the pharmaceutically acceptable carrier is an agent selected from the group consisting of excipient, solvent, emulsifier, suspending agent, decomposer, binding agent, stabilizing agent, chelating agent, diluent, gelling agent, preservative, lubricant, absorption delaying agent, liposome and combinations thereof.

12. The method of claim 1, wherein the pharmaceutical composition is in a dosage form for parenteral administration.

13. The method of claim 1, wherein the pharmaceutical composition is in a dosage form for oral administration.
